(19) United States
(12) Patent Application Publication
(10) Pub. No.: US 2026/0306108 A1
(43) Pub. Date: Oct. 8, 2026

(54) TOURNIQUET DEVICE

(71) Applicant: SHUH-TSAI YANG, Taipei City (TW)

(72) Inventor: SHUH-TSAI YANG, Taipei City (TW)

(21) Appl. No.: 19/172,628

(22) Filed: Apr. 7, 2025

(51) Int. Cl. A61B 17/132; A61B 17/00

(52) U.S. Cl. CPC A61B 17/1327; A61B 2017/00367; A61B 2017/00862; A61B 2503/40

(57) ABSTRACT

A tourniquet device is provided. The tourniquet device includes: a tourniquet base, having a base window portion and a first side portion and a second side portion; a driving shaft, having a shaft body, a first assembling portion and a second assembling portion; wherein the shaft body has a strap groove; a strap, having a fixed end and a free end; wherein the free end is wound to form an adjustable annular space and penetrates into the tourniquet base, and the free end is then wound into the strap groove and is rotatably linked to the driving shaft; a crank, having one end assembled to the first assembling portion of the driving shaft; a gear locking assembly, having a gear member and a pivot member; wherein rotating the crank drives the driving shaft to rotate synchronously, so that the annular space of the strap is tightened.

### FIELD OF THE DISCLOSURE

The present disclosure relates to the technical field of tourniquets, and in particular to a tourniquet device that is easy to apply.

### BACKGROUND OF THE DISCLOSURE

In conventional art, most tourniquet devices are tourniquet bands that wrap around the wound to achieve the purpose of stopping bleeding. However, winding the wound with a tourniquet is much more suitable for smaller wounds, but it is ineffective for stopping bleeding in larger wounds. Therefore, relevant industries have developed adjustable tourniquets, which are mainly to tie the tourniquet to the wound part, and then further tighten the tourniquet through a buckle, and adjust the tightness while tightening to achieve the effect of stopping bleeding.

However, the above method requires tightening the tourniquet manually, which requires greater force, so the application efficiency is poor. Moreover, it is difficult to control the force during the application process, resulting in too loose or tight problems.

### SUMMARY OF THE DISCLOSURE

The present disclosure provides a tourniquet device that can fine-tune the tightness of the strap through the crank mechanism so that the present disclosure can achieve the effect of hemostasis and is easy to apply.

In order to achieve the above-mentioned objectives, a tourniquet device is provided. The tourniquet device includes: a tourniquet base, having a base window portion and a first side portion and a second side portion extending from both sides of the base window portion; a driving shaft, having a shaft body, a first assembling portion and a second assembling portion; wherein the first assembling portion and the second assembling portion are disposed on both ends of the shaft body; wherein the shaft body has a strap groove and penetrates the first side portion and the second side portion, the first assembling portion and the second assembling portion are respectively exposed on an outer surface of the first side portion and the second side portion; a strap, having a fixed end and a free end on both ends; wherein the fixed end is fixed to the base window portion, the free end is wound to form an adjustable annular space and penetrates into the tourniquet base through the base window portion, and the free end is then wound into the strap groove of the driving shaft and is rotatably linked to the driving shaft; a crank, having one end assembled to the first assembling portion of the driving shaft; a gear locking assembly, having a gear member and a pivot member; wherein the gear member is assembled to the second assembling portion, one end of the pivot member is pivotally assembled to the second side portion and the other end of the pivot member is mutually meshed with the gear member; wherein rotating the crank drives the driving shaft to rotate synchronously, so that the annular space of the strap is tightened.

In certain embodiments, the first side portion and the second side portion have a first pivot hole and a second pivot hole respectively, and the first pivot hole and the second pivot hole respectively allow the shaft body of the driving shaft to pass through and be assembled pivotally.

In certain embodiments, the base window portion includes a plurality of windows allowing the free end of the strap to pass through.

In certain embodiments, the first assembling portion of the driving shaft has an assembling groove; wherein the assembling groove and one end of the crank respectively have a first assembling hole and a third assembling hole corresponding to each other; wherein a first assembling member passes through the first assembling hole and the third assembling hole, so that the crank is assembled to the assembling groove of the first assembling portion.

In certain embodiments, the gear locking assembly has a second assembling member, and the gear member and the second assembling portion have a fourth assembling hole and a second assembling hole corresponding to each; wherein the second assembling member passes through the fourth assembling hole and the second assembling hole, so that the gear member and the second assembling portion are assembled together and can be rotated.

In certain embodiments, the gear member has a plurality of gear cavities, and one end of the pivot member has a gear portion compatibly engaged with the plurality of gear cavities.

In certain embodiments, the gear locking assembly further includes an elastic member; one end of the elastic member is assembled to the pivot member, and the other end is assembled to the second side portion through the pivot member. Through the elastic member, the pivot member can pivotally move up or down.

In certain embodiments, a recording portion is disposed transversely between the first side portion and the second side portion.

### BRIEF DESCRIPTION OF THE DRAWINGS

The described embodiments may be better understood by reference to the following description and the accompanying drawings in which:

FIG. 1 is an exploded perspective schematic view of the present disclosure;

FIG. 2 is an assembled perspective schematic view of the present disclosure;

FIG. 3 is an assembled perspective schematic view of another angel of the present disclosure;

FIG. 4 is a sectional schematic view of line IV-IV of FIG. 2 of the present disclosure;

FIG. 5 is a perspective schematic view of turning the crank to adjust the tightness of the strap of the present disclosure;

FIG. 6 is a planar schematic view of the present disclosure that the gear member and the pivot member mesh with each other; and

FIG. 7 is a planar schematic view of the gear member and the pivot member disengaging from each other.

### DETAILED DESCRIPTION OF THE EXEMPLARY EMBODIMENTS

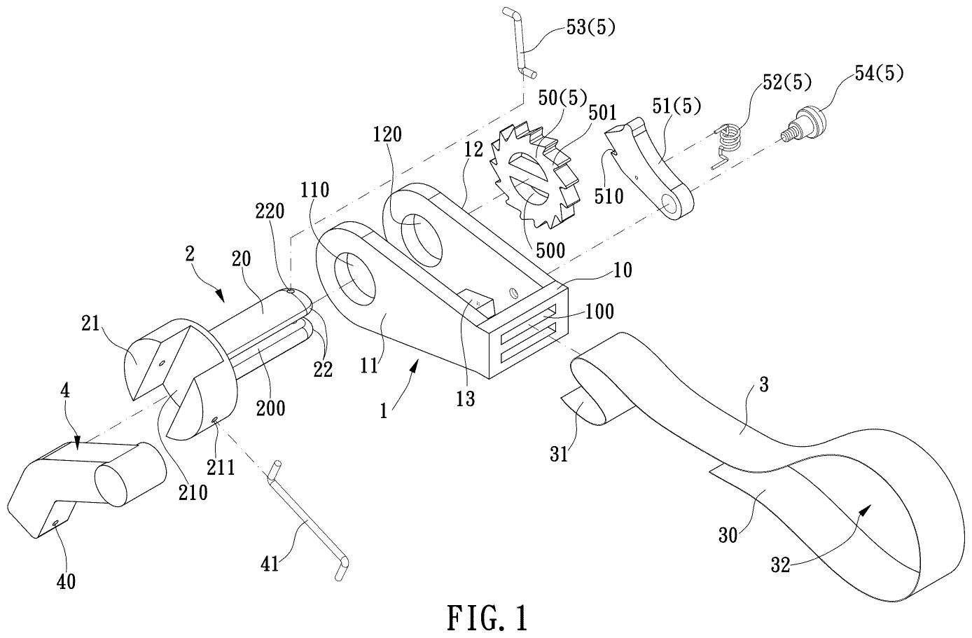

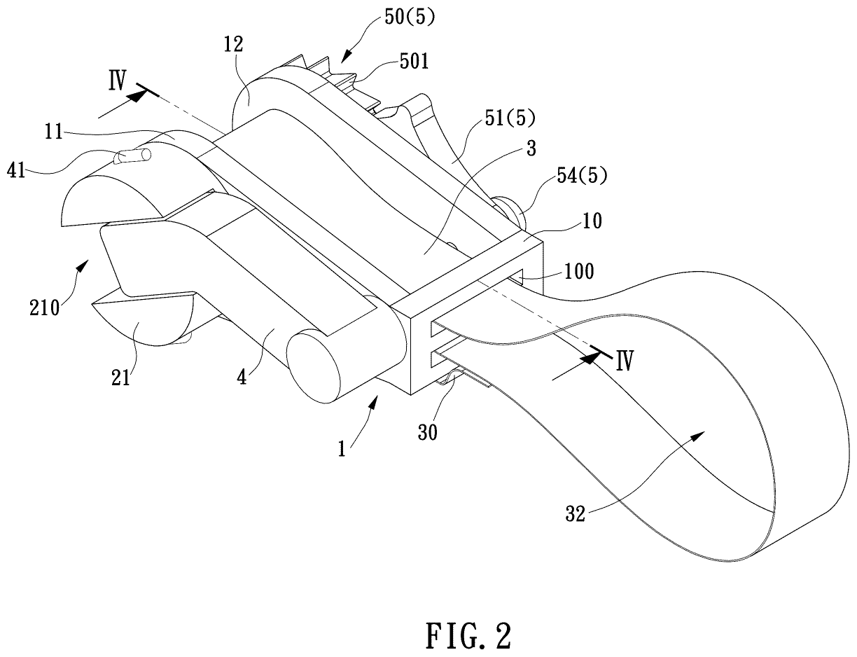

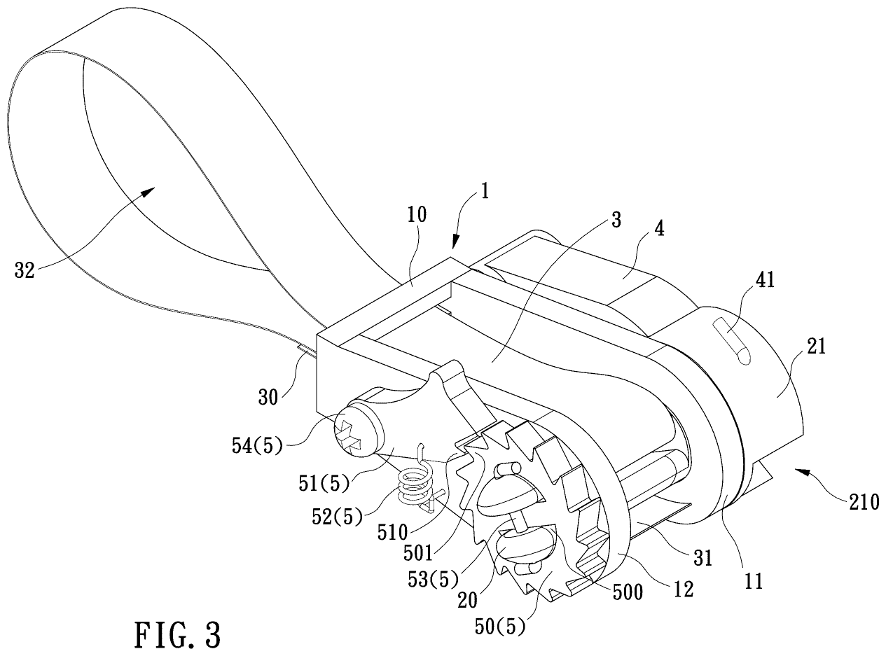

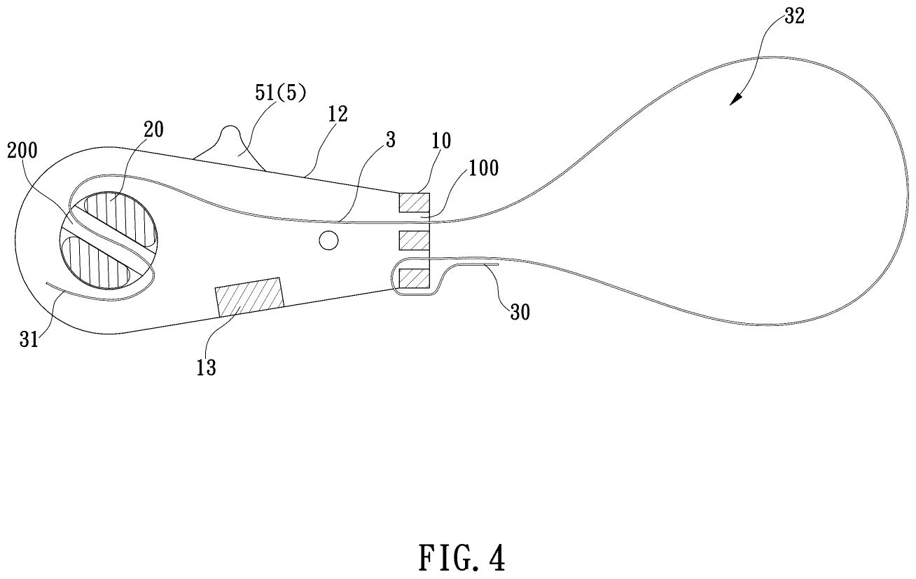

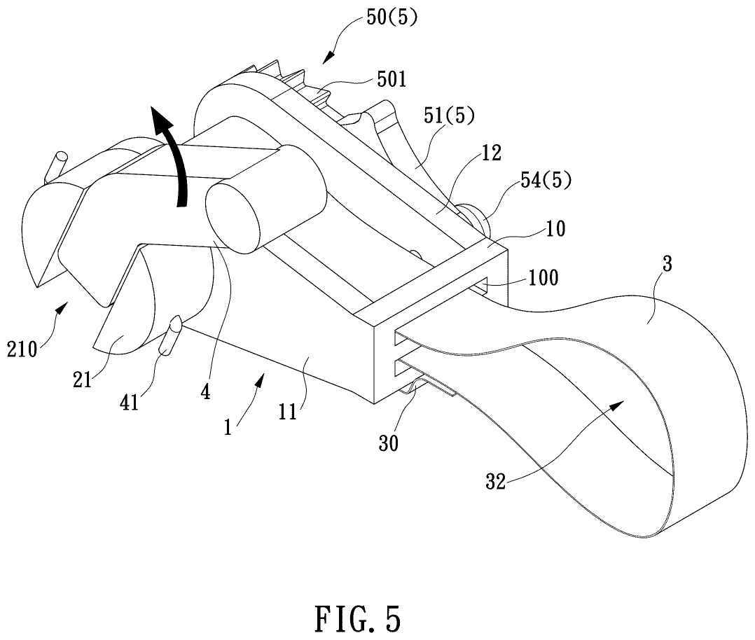

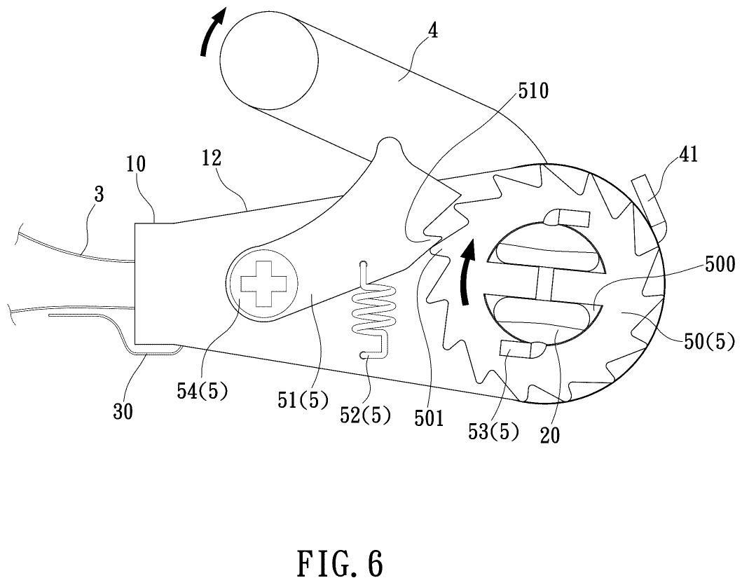

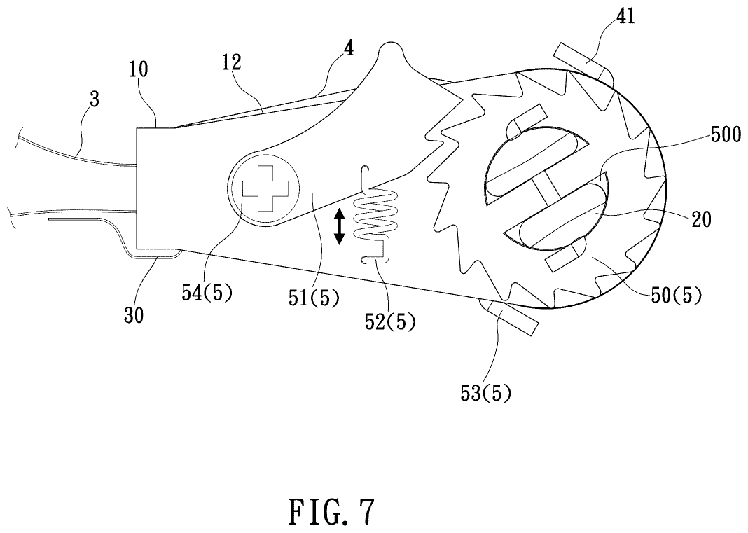

Referring to FIGS. 1-7, the present disclosure provides a tourniquet device, including a tourniquet base 1, a driving shaft 2, a strap 3, a crank 4, and a gear locking assembly 5.

The tourniquet base 1 is in C-shape and has a base window portion 10, a first side portion 11, and a second side portion 12 on both sides of the base window portion 10. The base window portion includes a plurality of windows 100. The first side portion 11 and the second side portion 12 respectively have a first pivot hole 110 and a second pivot hole 120. A recording portion 13 is disposed transversely between the first side portion 11 and the second side portion 12.

The driving shaft 2 has a shaft body 20, a first assembling portion 21 and a second assembling portion 22 disposed on both ends of the shaft body 20. The shaft body 20 has a strap groove 200. The shaft body 20 penetrates the first pivot hole 110 of the first side portion 11 and the second pivot hole 120 of the second side portion 12. The first assembling portion 21 and the second assembling portion 22 are respectively exposed outside of the first side portion 11 and the second side portion 12. Further, the first assembling portion 21 has an assembling groove 210. The assembling groove 210 and the second assembling portion 22 respectively have a first assembling hole 211 and a second assembling hole 220.

The strap 3 has a fixed end 30 and a free end 31 on both ends. The fixed end 30 is fixed to the base window portion 10, the free end 31 is wound to form an adjustable annular space 32. The free end 31 penetrates into the tourniquet base 1 through any of the window 120 of the base window portion 10, and the free end 31 is then wound into the strap groove 200 of the driving shaft 2 and is rotatably linked to the driving shaft 2.

The crank 4 has a third assembling hole 40 on one end corresponding to the first assembling hole 211 of the assembling groove 210. A first assembling member 41 passes through the first assembling hole 211 and the third assembling hole 40, so that the crank 4 is assembled to the assembling groove 210 of the first assembling portion 21.

The gear locking assembly 5 has a gear member 50, a pivot member 51, an elastic member 52, a second assembling member 53, and a pivot member 54. The gear member 50 has a fourth assembling hole 500 corresponding to the second assembling portion 22. The second assembling portion 22 of the driving shaft 2 passes through the fourth assembling hole 500, and the second assembling member 53 passes through the second assembling hole 220 of the second assembling portion 22, so that the gear member 50 and the second assembling portion 22 of the driving shaft 2 are assembled together and can be rotated together.

Further, the gear member 50 has a plurality of gear cavities 501 on the end surface. One end of the pivot member 51 is pivotally assembled to the second side portion 12 through the pivot member 54, and the other end of the pivot member 51 has a gear portion 510 meshed with one of the plurality of gear cavities 501. One end of the elastic member 52 is assembled to the pivot member 51, and the other end is assembled to the second side portion 12. Through the elastic member 52, the pivot member 51 can pivotally move up or down.

The present disclosure provides the tourniquet device for animals. The annular space 32 wound by the strap 3 is applied around the animal's wound, and the crank 4 is rotated to drive the driving shaft 2 and the gear member 50 to rotate synchronously. Through the rotation of the driving shaft 2, the annular space 32 of the strap 3 is gradually tightened to stop bleeding from the animal's wound, and the hemostasis time can be recorded in the recording portion 13 of the tourniquet base 1.

It is worth mentioning that when the gear member 50 rotates, one of the gear cavities 501 and the gear portion 510 of the pivot member 51 are engaged with each other, so as to fix the tightness of the annular space 32 of the strap 3. Moreover, the pivot member 51 forms an upper and lower pivot swing through the elastic member 52. Therefore, no matter how the gear member 50 rotates, the gear portion 510 of the pivot member 51 and the gear cavities 501 of the gear member 50 can always maintain a meshing relationship to prevent the driving shaft 2 from idling. When the strap 3 is to be removed or replaced, the pivot member 51 is moved to release the engagement between the pivot member 51 and the gear member 50, and the strap 3 can be pulled out from the strap groove 200 and the base window portion 10. If medical personnel require the emergency treatment, the strap 3 can be directly cut with a tool (such as scissors) to achieve the effect of quickly removing the strap 3.

With the structure mentioned above, the present disclosure can fine-tune the tightness of the annular space 32 of the strap 3 by simply rotating the crank 4. The operation is easy and there is no need to tie a knot on the strap 3. The strap 3 will not be entangled with the animal's fur, which can significantly reduce the animal's discomfort.

In addition, the conventional tourniquets are only single used to tighten the hemostatic area. If the tightness of the tourniquet needs to be adjusted, the tourniquet needs to be loosened and then re-tied manually, which is quite inconvenient to apply.

However, as shown in the present disclosure, if the tightness of the annular space 32 of the strap 3 is to be adjusted, it only requires to move the pivot member 51 to release the meshing engagement between the pivot member 51 and the gear member 50, pull the strap 3 out of the strap groove 200 and the base window portion 10, and then turn the crank 4 to re-tighten the annular space 32 of the strap 3. There is no need to re-tie the tourniquet manually as the conventional tourniquet, which is easier to operate.

What is claimed is:

1. A tourniquet device, comprising:
a tourniquet base, having a base window portion, a first side portion, and a second side portion; wherein the first side portion and the second side portion are disposed on both sides of the base window portion;
a driving shaft, having a shaft body, a first assembling portion, and a second assembling portion; wherein the first assembling portion and the second assembling portion are disposed on both ends of the shaft body; wherein the shaft body has a strap groove and penetrates the first side portion and the second side portion, the first assembling portion and the second assembling portion are respectively exposed on an outer surface of the first side portion and the second side portion;
a strap, having a fixed end and a free end on both ends; wherein the fixed end is fixed to the base window portion, the free end is wound to form an adjustable annular space and penetrates into the tourniquet base through the base window portion, and the free end is then wound into the strap groove of the driving shaft and is rotatably linked to the driving shaft;
a crank, having one end assembled to the first assembling portion of the driving shaft; and
a gear locking assembly, having a gear member and a pivot member; wherein the gear member is assembled to the second assembling portion, one end of the pivot member is pivotally assembled to the second side portion, and the other end of the pivot member is mutually meshed with the gear member;
wherein rotating the crank drives the driving shaft to rotate synchronously, so that the annular space of the strap is tightened.

2. The tourniquet device according to claim 1, wherein the first side portion and the second side portion have a first pivot hole and a second pivot hole respectively, and the first pivot hole and the second pivot hole allow the shaft body of the driving shaft to pass through and be assembled pivotally.

3. The tourniquet device according to claim 1, wherein the base window portion includes a plurality of windows allowing the free end of the strap to pass through.

4. The tourniquet device according to claim 1, wherein the first assembling portion of the driving shaft has an assembling groove; wherein the assembling groove and one end of the crank respectively have a first assembling hole and a third assembling hole corresponding to each other; wherein the first assembling hole and the third assembling hole are assembled through a first assembling member, so that the crank is assembled to the assembling groove of the first assembling portion.

5. The tourniquet device according to claim 1, wherein the gear locking assembly has a second assembling member, the second assembling member has a second assembling hole, and the gear member has a fourth assembling hole corresponding to the second assembling portion; wherein when the second assembling portion passes through the fourth assembling hole, the second assembling member passes through the second assembling hole, so that the gear member and the second assembling member are assembled together and move synchronously.

6. The tourniquet device according to claim 1, wherein the gear member has a plurality of gear cavities, and one end of the pivot member has a gear portion compatibly engaged with the plurality of gear cavities.

7. The tourniquet device according to claim 6, wherein the gear locking assembly further includes an elastic member, one end of the elastic member is assembled to the pivot member, and the other end is assembled to the second side portion through the pivot member.

8. The tourniquet device according to claim 1, wherein a recording portion is disposed transversely between the first side portion and the second side portion.
